(19) United States
(12) Patent Application Publication
(10) Pub. No.: US 2023/0255686 A1
(43) Pub. Date: Aug. 17, 2023

(54) ELECTROMAGNETIC ENERGY DELIVERY APPARATUS AND METHOD

(71) Applicant: Emblation Limited, Alloa (GB)

(72) Inventors: Eamon McErlean, Alloa (GB); Gary Beale, Alloa (GB)

(73) Assignee: Emblation Limited, Alloa (GB)

(21) Appl. No.: 18/017,157

(22) PCT Filed: Jul. 19, 2021
(86) PCT No.: PCT/EP2021/070177

§ 371 Date: Jan. 20, 2023

(30) Foreign Application Priority Data
Jul. 23, 2020 (GB) 2011414.6

(51) Int. Cl. A61B 18/18; H03F 3/19; H03G 3/00

(52) U.S. Cl. CPC A61B 18/1815; H03F 3/19; H03G 3/004; A61B 2018/00702; H03G 2201/103

(57) ABSTRACT

An electromagnetic energy delivery apparatus comprises: an amplifier; an amplifier input configured to provide to the amplifier a signal to be amplified; bias circuitry configured to provide a bias signal to the amplifier, wherein amplifying of the input signal by the amplifier is dependent on the bias signal provided by the bias circuitry; an amplifier output configured to provide an output signal comprising an amplified version of the input signal, for providing energy delivery to a radiating element to produce electromagnetic radiation; and a controller configured to control operation of the bias circuitry to provide a time- varying bias signal thereby to provide a desired time variation of the output signal.

The present invention relates to electromagnetic energy delivery apparatus and methods, for example apparatus or methods used to deliver pulsed or other microwave or r.f. radiation to a patient or other subject for treatment purposes.

### BACKGROUND

In most energy ablation systems, the energy is delivered from an energy generator, via a connecting cable, to a radiating applicator that transfers the energy into the tissue. In these applicators, the radiating element is surrounded by tissue or is placed in contact with the tissue. For such systems, the typical practice is to deliver energy for a treatment lasting typically anywhere from 1-20 minutes to raise the temperature of tissue greater than 43-45° C. for example to around or between 60° C., 70° C. or greater, such that necrosis occurs within the desired ablation zone. The energy may be delivered to have an amplitude or pulse width-modulated duty cycle to ensure the required level of energy is maintained or controlled for the duration of the energy release.

These known types of electromagnetic generator systems are designed to destroy diseases or unwanted tissue and are not designed to deliver treatments in a fundamentally immune responsive way, in concert with the adaptive or innate biological immune systems. Immune response may comprise for example : upregulation or down regulation of signalling; suppression or promotion of cell type growth, induction of apoptosis, modulation of cellular membrane.

In contrast with traditional treatments, a feature of immune response optimised treatments is the synergistic control of energy application in the temporal micro-scale that correlate with optimum immune response in diseased or abnormal tissues. Examples of immune response-related treatment systems and methods are described in WO 2019/239160, the contents of which are incorporated by reference.

Known electromagnetic energy generators are constructed using RF/Microwave energy circuits often comprising solid-state devices constructed using transistor amplifier technology. These devices are setup to operate in different amplifier classes depending upon the design of the amplifier and semiconductor technology of device used.

For a Class A amplifier, 100% of the input signal is used (conduction angle = 360°). The active element is conducting all of the time and this topology enables high amplifier gain. Class-A amplifiers are however inefficient in achieving this gain with a maximum theoretical efficiency of 25% obtainable using typical configurations. This inefficiency results in a lot of energy lost to heat to achieve the desired amplification gain.

In a class-B amplifier, the active device conducts for 180 degrees of the cycle. To avoid signal distortion two devices may be used. Each device conducts for one half (180°) of the signal cycle however some distortion may still exist in the region where the signals are joined termed as “cross-over distortion”. Class-B amplifiers have a higher theoretical efficiency of 75 \+% however this comes at the cost of low amplifier gain. Class-B amplifiers are most useful for pulsed signal amplification with low duty cycles requiring low average current.

To avoid cross-over distortion a scheme called Class AB may be employed to remove the cross-over distortion region where devices are not completely off. In this case the quiescent current (the current through both devices when there is no signal) is set to control the level of distortion. Class AB offers an efficiency between Class A and Class B depending upon design trade-offs.

There are various other amplifier classes, for example C, D, E, F, G, H, I, S, T, that offer efficiency/signal integrity trade-offs based upon controlled high frequency switching, for example, Class E amplifiers use LC resonant circuits similar to class C amplifiers, but in a class E amplifier the active device becomes a switch. Combinations of classes may also be employed such as Class AB/F etc.

As many medical applications only require the energy delivery to be used for thermal and high frequency oscillation effects, design trade-offs against communication or signal applications may be made to improve efficiency. However, the majority of packaged integrated circuit (RFIC/MMIC) devices are supplied designed for the communication market and are optimised for these applications. Medical applications would benefit most from a topology akin to Class B but with more gain, or a PWM pulsed class that could approach maximum efficiency. These amplifier Class efficiencies are concerned with signal conversion and output signal quality/integrity however for low duty usage the active biasing arrangements can become another source of inefficiency.

Typically, amplifiers are supplied by energy from biasing circuits which delivers high current, low voltage electrical power to enable the conversion from DC power to microwave energy often at a level of efficiency which may be 30-40% depending upon the biasing scheme employed. The biasing being designed to control gain or efficiency or some other aspect of amplifier performance.

The biasing for RF or microwave power circuits operates in a quiescent state and the transistor/amplifier circuits are energised in readiness for a signal that is amplified to be output as high frequency energy. This signal may be continuous or pulse modulated (via switched input) to achieve a particular average or RMS output level. During the quiescent standby state, the transistor amplifier draws current which results in waste energy in the form of heat. Whilst this is often not a significant issue in mains powered units, or in communication applications that are permanently running it presents as a continuous drain to a battery powered device. In such a device used for a very brief treatment protocol lasting a few seconds the standby usage may dominate overall battery usage over a prolonged period.

### SUMMARY

According to a first aspect of the present invention there is provided an electromagnetic energy delivery apparatus comprising:
an amplifier;
an amplifier input configured to provide to the amplifier a signal to be amplified;
bias circuitry configured to provide a bias signal to the amplifier, wherein amplifying of the input signal by the amplifier is dependent on the bias signal provided by the bias circuitry;
an amplifier output configured to provide an output signal comprising an amplified version of the input signal, for providing energy delivery to a radiating element to produce electromagnetic radiation; and
a controller configured to control operation of the bias circuitry to provide a time-varying bias signal thereby to provide a desired time variation of the output signal.

The controller may be configured to reduce or switch off the bias signal during time periods when no output signal is desired thereby to reduce power consumption.

The apparatus may comprise a medical apparatus for applying r.f. or microwave radiation to a subject, and/or the input signal comprises an r.f. or microwave-frequency signal, and/or the amplifier output may be arranged to provide the output signal to a device comprising an antenna or other radiating element, optionally comprising or forming part of a hand-held r.f. or microwave applicator.

The apparatus may comprise a medical apparatus for providing a treatment to a subject, and the controller controls the time-varying bias signal to provide a corresponding desired time-varying treatment to the subject.

The desired variation of the output signal may comprise a series of pulses, and the controller is configured to reduce or switch off the bias signal for times between the pulses.

The input signal may be continuous and/or non-pulsed and/or may have a different time dependence to the bias signal and/or a different modulation to the bias signal, and a desired pulsed output signal may be obtained by control of the time variation of the bias signal.

The apparatus may be configured to provide energy delivery during a series of energy delivery periods.

The controller may be configured to reduce or switch off the bias signal during interval(s) between the energy delivery periods.

For each energy delivery period, the controller may be configured to control the bias signal to provide at least one desired property of the output signal, for example a desired total energy and/desired power for the energy delivery period.

Each energy delivery period may be divided into a plurality of sub-periods. The controller may be configured to control, for example, repeatedly switch on and off or increase and decrease, the bias signal to provide at least one desired property of output signal for the sub-period and/or the energy delivery period, for example a desired total energy and/desired power for the sub-period and/or for the energy delivery period.

For each of the energy delivery periods the energy may be delivered as a series of pulses of the output signal.

For each pulse, the controller may be configured to control the bias signal to provide at least one desired property of the pulse, for example a desired energy and/or desired power and/or desired duration of the pulse.

The controller may be configured to control the bias signal to provide pulse width modulation, for example a series of pulses each with a respective desired width.

The bias circuitry may comprise a transistor and/or switch, and the controller may control operation of the transistor and/or switch to provide the time-varying bias signal.

The amplifier may comprise negative and positive bias inputs. The bias circuitry may be configured to provide a negative voltage to the negative input and a positive voltage to the positive input, and the bias circuitry and/or controller may be configured to offset the application of the negative and positive voltages to the inputs, optionally so that the negative voltage is removed/reduced after and/or applied/increased before the positive voltage.

The amplifier may form part of a gain stage and/or the apparatus may comprise a further gain stage connected to the gain stage and comprising a further amplifier and further bias circuitry. The controller may be configured to control operation of the further bias circuitry to provide a time-varying bias signal for example thereby to provide a desired time variation of an output signal from the further amplifier.

The controller may be further configured to control power to at least one further component or device, and/or to switch off or reduce power to such at least one further component or device in a power saving mode, and/or to provide higher, operational power to the at least one further component or device in an operational mode.

In the operational mode, the controller may be configured to reduce or switch off the bias signal during time periods when no output signal is desired thereby to reduce power consumption in the operational mode.

The controller may be configured to control the bias signal in dependence on input from a sensor and/or in dependence on input from a user.

The controller may be configured to reduce or switch off the bias signal to enter a low power mode in response to the sensor and/or user input.

The controller may control the time-varying bias signal to reduce heat and/or thermal burden, for example to reduce or prevent overheating.

The apparatus may comprises a medical apparatus for providing treatment to a patient or other subject.

The apparatus may comprise or be for connection to a display device and or analogue-to-digital and/or digital-to-analogue converter.

The output signal may have power, or may be such that the resulting electromagnetic radiation has power, in a range 1-50 W, optionally 8 W-10 W, 2 W-5 W or 3 W-8 W.

The input signal may have a frequency in a range of 0.1 GHz to 300 GHz.

The controller may be configured to control the bias signal to provide amplitude modulation and/or pulse width modulation of the output signal, optionally amplitude modulation and/or pulse width modulation with a modulation frequency in a range 1 Hz to 500 KHz.

The input signal may be frequency modulated, optionally wherein the frequency modulation is in a range 1 to 500 KHz.

The controller may be configured to control the bias signal so that the output signal is modulated in accordance with a pulse width modulation (PWM) or an on/off keying (OOK) modulation scheme.

The apparatus may comprise an antenna or other radiating element that receives the output signal and outputs corresponding electromagnetic radiation.

In a further aspect, which may be provided independently, there is provided an electromagnetic energy delivery system comprising:
a signal generator for generating r.f. or microwave or other signals having a desired frequency;
an apparatus as claimed or describer herein, configured so that the signals from the generator are provided to the amplifier input of the apparatus; and
a radiating element arranged to receive output signals from the amplifier output of the apparatus and to produce corresponding electromagnetic radiation

In another aspect, which may be provided independently, there is provided a method of controlling operation of an electromagnetic energy delivery apparatus comprising controlling a bias signal applied to an amplifier of the apparatus thereby to provide a desired time variation of an output signal of the apparatus.

Further aspects, which may be provided independently, relate to various beneficial methods and delivery profiles relating to the control of an RF or microwave amplifier based medical device.

Combinations of pulse regimes may be incorporated with bias control to eliminate power usage outside of treatment energy pulses and/or treatment dose periods. This may provide, firstly, a reduction in amplifier quiescent current heating placing less burden on the thermal management system and, secondly, a reduction in standby energy usage in battery powered devices. Indirectly reduction in thermal losses can prolong the lifetime of many electronic components.

Applied energy may be in the form of a continuous oscillating electromagnetic wave (CW) at a fixed frequency or modulated (variable frequency). The frequency could range from 1 MHz to 300 GHz but preferentially may be in the microwave range from 0.9 GHz to 60 GHz.

Pulse regimes may include amplitude control of signal energy (AM pulsing) and pulse width modulation control (PWM) and on/off keying (OOK).

Modulation schemes may include pulse modulation rates (1-100 kHz) or frequency modulation rate (1-500 kHz)

Treatment durations may be single shot or multiple shot or continuous energy dose for a treatment session. A single shot may be one thousandth of a second, one second, two seconds, or any time duration up to twenty seconds or up to one or two or ten minutes followed by cessation of energy delivery. Optionally a single shot may be two-three seconds, at a power level required for the treatment.

A multiple shot may be a repeat of a single shot as described above for a number of treatment doses from one to one hundred or one thousand doses in a treatment session. Optionally, multiple shots may be five-ten times during a treatment dose.

A continuous dose may be a fixed level of energy or a modulated level of energy during a treatment session. This continuous energy delivery may be pulsed modulated any suitable number of times, for example one or five or fifty times or one hundred or one thousand or ten thousand or one hundred thousand times a second, or between 1 and 100,000 times a second, optionally between 50 and 10,000, further optionally between 100 and 1,000 times per second, during the treatment session. Optionally, continuous energy delivery may be pulsed modulated at one thousand times per second (1 kHz).

In a further aspect, which may be provided independently, there is provided an electromagnetic treatment method or system that which utilises an energy efficient biasing scheme to increase efficiency and/or reduce thermal losses and/or prolong battery powered microwave applications.

The electromagnetic treatment method or system may use or comprise an energy generator system, and may be fully integrated or may include cabling and applicator which provides a transmission path for electromagnetic energy from the generator system to a recipient device, for example a contact or radiating applicator that transfers the energy into biological tissue.

The method may comprise, or the system may be configured to operate by, applying power to the circuit to correlate with delivering energy in specific temporal dosage profiles or protocols. The power may be removed at other times, optionally all other times, to facilitate a hibernation state that uses minimal to no power until the next application of energy is required thus reducing heating or standby power losses.

One or more aspects may provide suspending or reducing power to a circuit except during times that the energy is required, such times corresponding to a specific treatment protocol, for example relating to brief treatments.

According to a further aspect, which may be provided independently, there is provided an electromagnetic energy delivery system that actively controls energy usage to efficiently consume energy only during treatment delivery protocols. This may optionally be achieved by bias control and/or hierarchal control and/or thermal management control. Optionally, the energy may be consumed and/or delivered and/or used only during sub-cycle times and/or during sub-cycle constituent pulsed times.

Power management schemes may be provided that are synchronised and/or work in concert with energy delivery and/or treatment periods, optionally said periods may each have a duration in the order of seconds.

Features in one aspect may be provided as features in any other aspect, for example method features may be provided as apparatus features and vice versa.

### BRIEF DESCRIPTION OF THE DRAWINGS

FIG. 1 is a diagrammatic illustration of an electromagnetic energy delivery according to an embodiment;

FIG. 2 is a diagrammatic illustration of amplifier biasing arrangements;

FIG. 3 is a diagrammatic illustration of an electromagnetic energy treatment profile that includes periodic energy delivery intervals including pulse width modulated energy packets, controlled by modulated control of the amplifier bias;

FIG. 4 is a diagrammatic illustration of an electromagnetic energy treatment profile that includes pulse-width modulated energy delivery intervals comprising pulse-width modulated energy packets, controlled by modulated control of the amplifier bias;

FIG. 5 is a diagrammatic illustration of an electromagnetic energy treatment profile that includes periodic energy delivery intervals of energy packets (containing pulse-width modulated energy) and frequency modulated energy; and

FIG. 6 is a diagrammatic illustration of an electromagnetic energy treatment profile that includes periodic energy delivery intervals of energy packets (containing pulse width modulated energy) continuous-wave energy and/or amplitude modulated energy and/or frequency modulated energy.

### DETAILED DESCRIPTION

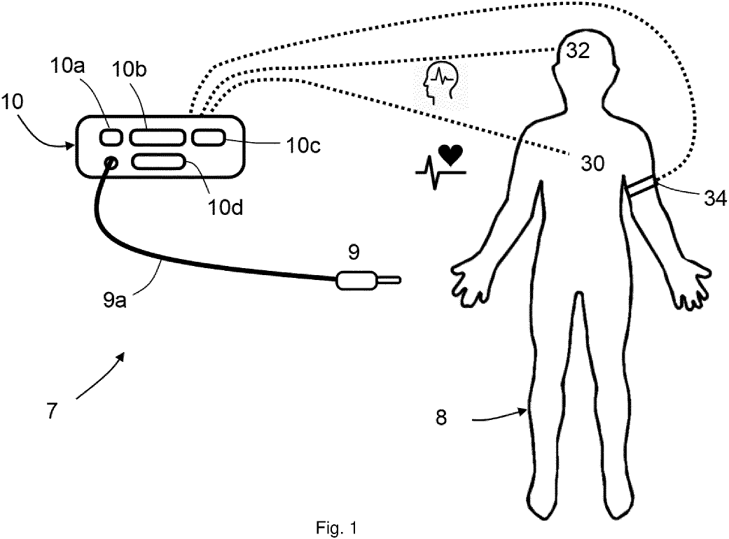

Referring initially to FIG. 1 there is shown an electromagnetic energy delivery system generally designated 7 for stimulating and/or inhibiting an immune response in a patient or other subject generally designated 8, and/or for performing ablation or heating or other procedure.

The system 7 includes a controlled electromagnetic energy generator apparatus generally designated 10, an electromagnetic energy applicator 9 including one or more antennas for radiating and/or applying electromagnetic energy to the subject 8, and a cable 9a for transmitting electromagnetic energy from the controlled electromagnetic energy generator apparatus 10 to the electromagnetic energy applicator 9.

The controlled electromagnetic energy generator apparatus 10 includes an electromagnetic energy source 10a, a controller 10b, a memory 10c, and a user interface 10d. The memory 10c contains instructions which, when executed by the controller 10b, cause the controller 10b to control the electromagnetic energy source 10a to emit electromagnetic energy according to one or more treatment profiles. The one or more treatment profiles may, for example, be stored in the memory 10c. Additionally or alternatively, the one or more treatment profiles may be manually input via the user interface 10d.

The cable 9a includes, or takes the form of, a waveguide for transmitting the electromagnetic energy emitted by the electromagnetic energy source 10a to the one or more antennas of the electromagnetic energy applicator 9. The cable 9a may include, or take the form of, a co-axial cable. The cable 9a may be flexible or rigid.

In use, the electromagnetic energy applicator 9 is held adjacent to, and/or in contact with, the subject 8 and the controller 10b controls the electromagnetic energy source 10a to emit electromagnetic energy according to one or more of the treatment profiles for delivery of electromagnetic energy to the subject 8 according to the one or more of the treatment profiles via the cable 9a and the electromagnetic energy applicator 9. In an exemplary embodiment, the controlled electromagnetic energy generator apparatus 10 may be configured for applying microwave energy to the subject 8 and the electromagnetic energy applicator 9 may be a microwave applicator. In such an embodiment, the electromagnetic energy source 10a may be configured to emit microwave energy and the cable 9a may be configured to transmit the emitted microwave energy to the one or more antennas of the microwave applicator 9.

Any suitable controller 10b may be used and a memory 10c may or may not be provided. In certain embodiments, the controller 10b comprises a programmed or programmable general purpose processor, or dedicated processor, or dedicated control circuitry for example one or more ASICS or FPGAs.

The electromagnetic energy source 10 includes one or more amplifiers 20 for amplifying generated signals before they are provided to the applicator 9 via cable 9a.

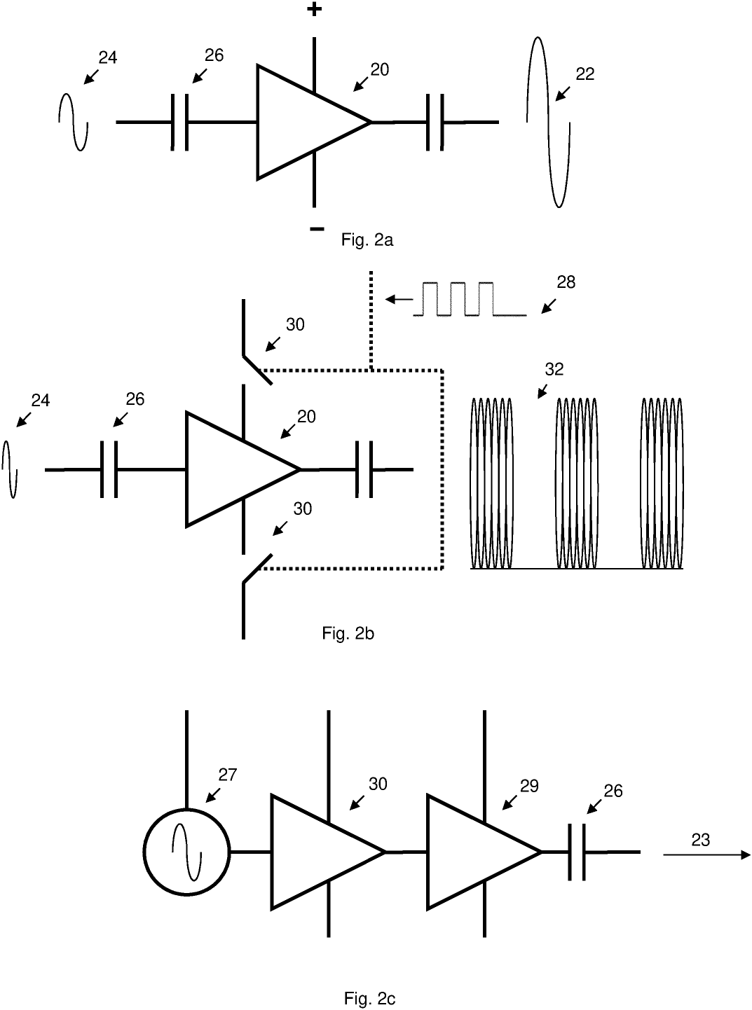

A number of amplifier biasing arrangements included in various embodiments according to FIG. 1, and operating under control of the controller 10b, are illustrated in FIGS. 2a, 2b and 2c. In the first basic arrangement of FIG. 2a an amplified signal 22 having a specified gain is created when bias is supplied to an amplifier 20 via bias circuitry (not shown), under control of the controller 10b, and an alternating (sinusoidal or other) signal 24 is capacitively coupled via a capacitive arrangement 26, e.g. a capacitor, into the amplifier 20 to output an amplified version. In this instance the amplifier 20 is represented by the symbol for a gain stage however this may be understood, in some embodiments, to comprise a transistor or arrangement of transistors or MMICs or hybrid integrated circuits intended to provide the function of an amplifier.

In FIG. 2b a control signal 28 in the form of a modulation (pulse train) is applied, under control of the controller 10b, to control the bias of the amplifier 20, by means of bias circuitry in the form of a switching arrangement 30 realised by using at least one other transistor or dedicated switch device. This causes the amplifier 20 to stop amplifying during the time that the bias is switched off. The resultant is an output signal 32 of amplified energy that possesses the same modulation temporal properties as the control signal 28.

The switching arrangement 30 operates by deactivating the positive and negative bias voltages during the times energy output is not required. Some devices according to possess a standby selection feature however these may still permit energy to be used by placing the device in a low power mode whereas the approach herein eliminates any residual energy usage. Some devices also require application of negative bias for protection and this may be arranged to be removed last and replaced first as a related function of the control signal.

The control signal 28 may comprise bias modulation of any suitable type, and a time constant of the control signal may be low/slow (in the order of seconds) or high/fast in the order of a hundred or thousands of times per second or more and depending upon the switch speed of the amplifier device 20. This switching rate if too high may have detrimental effects such as transients (L di/dt) and heating losses (dv/dt).

The mode of operation described possesses similarities to Class E operation, however rather than control the bias to reshape the amplified signal in order to recreate the sinusoidal integrity by control of the harmonic content, according to embodiments the power output (e.g. the amplified signal) can be modulated to correspond to a medical treatment profile for a medical instrument. Once treatment has finished the amplifier bias can be removed or otherwise modified to enter a fully hibernated state until the next requirement of energy. This can, for example, be as long as the next treatment duration or short as the next fundamental pulse-width modulation component or any combination thereof.

In FIGS. 2c, a multistage device is illustrated with, an oscillator 27 and two distinct gain stages, 29, 30. The same or similar approach to that described in relation to FIGS. 2a and 2b may be used in the embodiment of FIG. 2c in applying control signals, under control of the controller 10b, via bias circuitry (not shown) to bias one or more or all stages 29, 30 to remove or reduce the power of the signal during a desired period, for example during a modulation off state. In a medical application unlike a communication application a rise or fall time of tens or hundreds of milliseconds can be advantageously accommodated and incorporated within a treatment duration lasting in the order of seconds or minutes. This technique can also remove the requirement of an RF/Microwave switch that is often used to apply a modulation to an incoming low power amplifier signal for the purposes of pulse width modulation control. Simply opening such an input switch would remove the output energy, however the amplifier bias would operate at all times draining power from the circuit even when the amplifier output power is low or off, unlike in the embodiments of FIGS. 2a to 2c.

The amplified signals from the amplifiers of FIGS. 2a to 2c are provided to an antenna or other radiating element, for example to the one or more antennas of the microwave applicator 9, to generate corresponding electromagnetic radiation. In some embodiments one or more further components can be provided between the amplifier and the radiating element(s) if desired as well as or instead of the cable 9a or other transmitting or waveguide components, for example to provide additional processing of the amplified signals.

The bias circuitry for providing the bias signals to the amplifier can be any suitable circuity for providing desire voltages or other signals to the amplifier(s) with desired time-varying characteristics, as controlled by the controller.

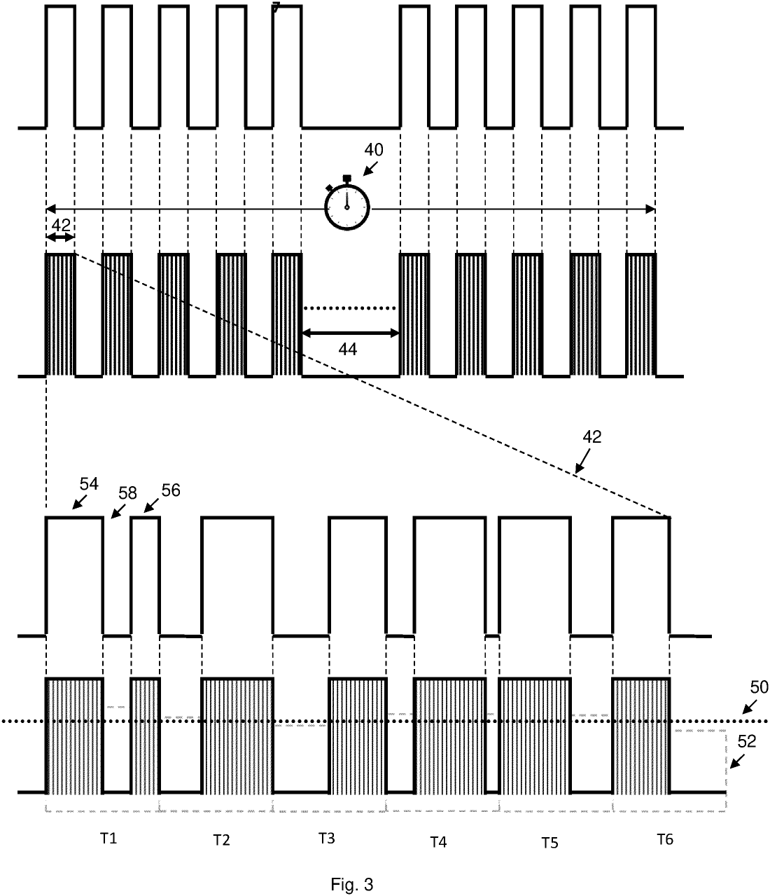

With reference to FIG. 3 an example of a treatment profile according to an embodiment is illustrated which represents time-variation of electromagnetic energy (for example, microwave or r.f. signals) applied to a patient or other subject. The treatment profile may for example be provided by the output signals 22, 32 of the embodiments of FIGS. 2a, 2b or 2c.

In the treatment profile of FIG. 3, there is a time duration 40 representing the overall treatment time. This may be in seconds, minutes or hours and may, for example, be between one and thirty minutes in some embodiments. Within this time duration 40 a number of energy delivery periods (or doses) 42 are provided. These periods 42 may, for example, be proportions or multiples of one second to five seconds, ten seconds, twenty seconds or any other period that is a proportion of the time duration 40. A fixed or variable number of these energy delivery periods 42 may be delivered and may include a treatment interval 44 where no or reduced energy is delivered and/or there is a break from treatment . The interval 44 can be between each energy delivery period 42 or may be a longer interval 44 (as illustrated schematically in FIG. 3) between a series of energy delivery periods 42. An example of this according to an embodiment would be a microwave treatment system that delivers ten Watts of energy for a period of two seconds and each energy delivery period is repeated five times, with this cycle being repeated for up to fifteen minutes.

Energy delivery is not continuous during each energy delivery period 42 in the embodiment of FIG. 3. One of the energy delivery periods (or doses) 42 is shown schematically in expanded form in FIG. 3 and it can be seen there may be provided a number of time-frames, also referred to as energy delivery sub-periods, and labelled T1 to T6, which together make up the energy delivery period. Each time frame may, for example, have a duration of a selected and/or pre-determined number of seconds or fractions of seconds.

Within each of these time frames T1-T6 the energy output may be repeatedly and variably pulse-width modulated using bias modulation, or otherwise varied, thereby switching on or off (or increasing decreasing) energy delivery to control the average or RMS power 50 (illustrated schematically in FIG. 3 by dotted line) delivered across the entire energy delivery period 42. This control may be via feedback of a measured sample of amplifier output power that would be compared to a set power reference and using a difference signal the bias control would be adjusted accordingly to maintain the required average or RMS power output 50. The difference signal may represent the proportion or duty of a full pulse width for each timeframe, for example ½ duty = 50% pulse width modulation = half power. It can be seen for example for time frame T1 there are two pulse periods 54, 56 of different lengths during which power is delivered, separated by a period 58 during which there is no power delivery.

It can be seen in FIG. 3 that the average or RMS power 52 (illustrated schematically in FIG. 3 by a dashed line) is different for some of the time frames/sub-periods T1-T6 than for some other of the time frames/sub-periods T1-T6. The bias applied can be controlled such that, even though there may be variation in the average or RMS or other measure of power or energy delivered for each time frame/sub-period T1-T6, the average or RMS or other measure of power or energy delivered for the whole energy delivery period 42 (and/or for all of the energy delivery periods 42 in combination) has a desired value or is in a desired range.

The controller may apply any suitable rules to control the average or RMS or other measure of power or energy delivered for each time frame/sub-period T1-T6 and/or for each energy delivery period 42 and/or for the energy delivery periods 42 in combination. For example, some treatments may desirably include different levels of energy delivery for different ones of the time frames or sub-periods e.g. T1-T6, for example increasing or decreasing with time or having some other desired profile. Alternatively or additionally the average or RMS or other measure of power or energy delivered for each time frame/sub-period e.g. T1-T6 may be varied based on a feedback or other control parameter, for example temperature, whilst ensuring that the average or RMS or other measure of power or energy delivered for the whole energy delivery period 42 (and/or for all of the energy delivery periods 42 in combination) is at a desired level or in a desired range.

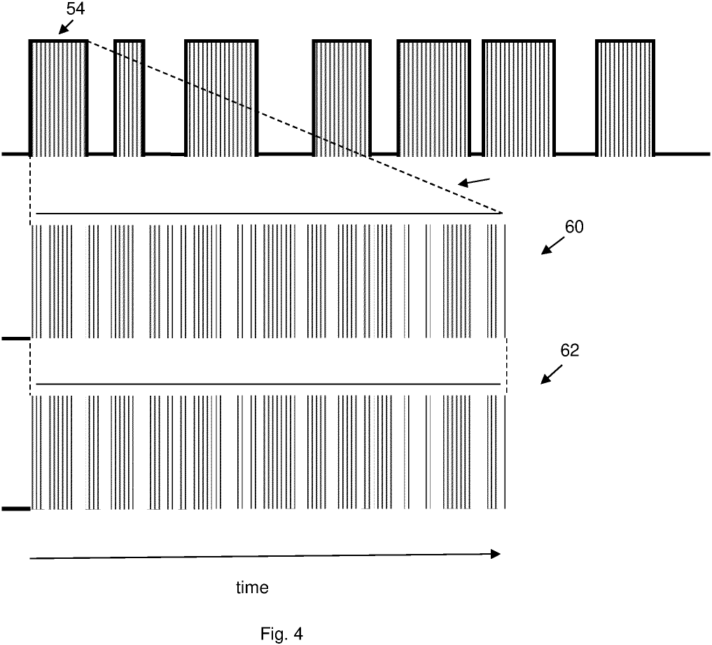

In variants of the embodiment of FIG. 3, the bias control modulation and the pulse width modulation may also be temporally equivalent. In FIG. 4, the pulse period 54 of timeframe T1 is expanded to illustrate schematically the individual pulses, represented by vertical lines in the lower part of FIG. 4, used to construct the energy delivery of pulse period 54. A level of pulsing (e.g. number of timing of pulses) may be applied as a control signal 60 to bias input(s) of the amplifier to provide a corresponding pulsed energy output 62 from the amplifier. Thus, the bias modulation may be divided down to the most fundamental pulse level required or supported by the amplifier to create the energy output. In this regard power is only used during each fundament pulse which may further save energy or reduce heating losses.

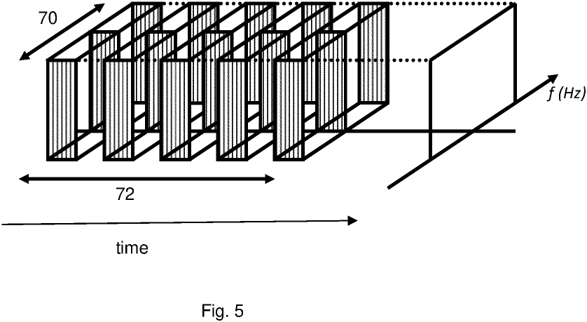

This is further developed in FIG. 5 where the signals can be frequency modulated 70 and delivered temporally 80; in this case a lower frequency signal is superimposed on a high frequency signal. This can be achieved as amplitude or frequency modulation or superposition of both resulting in the following in the embodiment of FIG. 5:-
a. Carrier, e.g. 8 GHz.
b. AM PWM modulation, e.g. 1-10 kHz, (bias control).
c. Frequency modulation bandwidth of 8 GHz carrier, e.g. 100-200 MHz.
d. Frequency modulation rate of 8 GHz carrier, e.g. 1-10 kHz.

For an amplifier with bias modulation control for a medical application this carrier may be any RF or Microwave frequency ranging, for example, from 100 MHz to 300 GHz. The bias signal modulation, e.g. the AM PWM modulation, may be any suitable frequency, for example from 1 Hz to 500 KHz. The Frequency modulation bandwidth of the e.g. 8 GHz carrier may, for example, be one or more octaves or from 1-2 GHz. The Frequency modulation rate of the carrier may, for example, be 1-500 kHz.

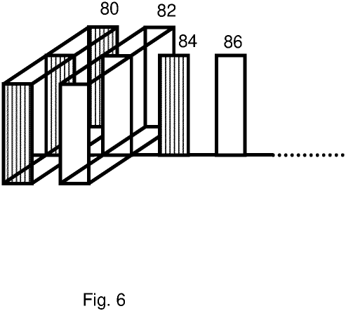

According to embodiments, these modulation schemes may be dynamically applied to be frequency modulated/pulse width modulated 80, frequency modulated continuous wave 82, fixed frequency/pulse width modulated 84 or fixed frequency 86 or any combination thereof as illustrated in FIG. 6, in which vertical lines indicate pulses schematically and blank boxes can indicate continuous wave signals.

The various bias control features may be used in a hierarchical control system according to embodiments, for example displays and power of a medical device may be turned off between use, for example if no movement or action is detected for a period of time. Below this, in some embodiments, various circuit parameters are activated and deactivated as required to save power such as backlights or cooling devices or power/voltage regulation or conversion circuits. Further again lower level components such as power amplifier integrated circuits or transistors may be switched off when treatment stops and further below this during treatment at times not required to achieve an average or RMS power these components such as amplifiers may be momentarily turned off. Further again below this at the fundamental pulse level components may be deactivated from consuming power at the end of each pulse.

It should be noted that this scheme has been discussed for application in a medical device to save power for example a battery powered device, portable, static or handheld. However, by way of saving power the same scheme may be used in certain embodiments to reduce heat or thermal burden of inefficient DC to high frequency energy conversion or DC to display illumination or any other conversion from DC power to an energy function.

The various bias control features may, alternatively or additionally, be applied to reduce heating effects to prevent overheating of a unit by only accumulating heat losses during use. This may, for example, apply to any device, e.g. battery powered or mains powered, where careful thermal control is required. For example, in medical devices limits my be placed on acceptable contact temperatures and reducing heating losses helps to ensure these limits are met.

Again, these heating losses may be managed by a hierarchical control system, for example displays and power of a medical device may be turned off between use to reduce heating, for example if no movement or action is detected for a period of time. Below this various circuit parameters are activated and deactivated as required to reduce heating such as backlights or cooling devices or power/voltage regulation or conversion circuits. Further again lower level components such as power amplifier integrated circuits or transistors may be switched off when a treatment finishes to reduce heating and further below this during treatment at times not required to achieve an average or RMS power these components such as amplifiers may be momentarily switched off to reduce heat losses. Further again below this at the fundamental pulse level components may be deactivated to reduce heating at the end of each pulse providing such switching rates do not intrinsically produce heating.

Control of bias may be provided by control of direct bias applied to a microwave amplifier or transistor device. Alternatively or additionally, control of bias may be obtained in certain embodiments by control of circuits that create or regulate the bias voltages, for example voltage regulator(s) or buck/boost conversion circuit(s) or any other control of a circuit level above or below this in hierarchy back to the power source.

In alternative embodiments, apparatus and methods described herein may be implemented as, or using, suitably modified versions of microwave or other electromagnetic energy delivery systems described in any one of WO 2018/037238, WO 2018/178659, WO 2019/239160 or WO 2020/049283, the contents of each of which are hereby incorporated by reference.

It will be understood that the present invention has been described above purely by way of example, and modifications of detail can be made within the scope of the invention. Each feature disclosed in the description, and (where appropriate) the claims and drawings may be provided independently or in any appropriate combination.

1. An electromagnetic energy delivery apparatus comprising:
an amplifier;
an amplifier input configured to provide to the amplifier a signal to be amplified;
bias circuitry configured to provide a bias signal to the amplifier, wherein amplifying of the input signal by the amplifier is dependent on the bias signal provided by the bias circuitry;
an amplifier output configured to provide an output signal comprising an amplified version of the input signal, for providing energy delivery to a radiating element to produce electromagnetic radiation; and
a controller configured to control operation of the bias circuitry to provide a time-varying bias signal thereby to provide a desired time variation of the output signal.

2. The apparatus according to claim 1, wherein the controller is configured to reduce or switch off the bias signal during time periods when no output signal is desired thereby to reduce power consumption.

3. The apparatus according to claim 1, wherein the apparatus comprise a medical apparatus for applying r.f. or microwave radiation to a subject, and/or the input signal comprises an r.f. or microwave-frequency signal, and/or the amplifier output is arranged to provide the output signal to a device comprising an antenna or other radiating element, optionally comprising or forming part of a hand-held r.f. or microwave applicator.

4. The apparatus according to claim 1, wherein the apparatus comprises a medical apparatus for providing a treatment to a subject, and the controller controls the time-varying bias signal to provide a corresponding desired time-varying treatment to the subject.

5. The apparatus according to claim 1, wherein the desired variation of the output signal comprises a series of pulses, and the controller is configured to reduce or switch off the bias signal for times between the pulses.

6. The apparatus according to claim 1, wherein the input signal is continuous and/or non-pulsed and/or has a different time dependence to the bias signal and/or a different modulation to the bias signal, and a desired pulsed output signal is obtained by control of the time variation of the bias signal.

7. The apparatus according to claim 1, wherein the apparatus is configured to provide energy delivery during a series of energy delivery periods.

8. The apparatus according to claim 7, wherein the controller is configured to reduce or switch off the bias signal during interval(s) between the energy delivery periods.

9. The apparatus according to claim 7 wherein, for each energy delivery period, the controller is configured to control the bias signal to provide at least one desired property of the output signal, wherein the desired property includes at least one of a desired total energy or a desired power for the energy delivery period.

10. The apparatus according to claim 7, wherein each energy delivery period is divided into a plurality of sub-periods, and, the controller is configured to control by repeatedly switching on and off or increase and decrease, the bias signal to provide at least one desired property of output signal for the sub-period and/or the energy delivery period, wherein the desired property includes at least one of a desired total energy or a desired power for the energy delivery period.

11. The apparatus according to claim 7, wherein for each of the energy delivery periods the energy is delivered as a series of pulses of the output signal.

12. The apparatus according to claim 7, wherein, for each pulse, the controller is configured to control the bias signal to provide at least one desired property of the pulse, wherein the desired property includes at least one of a desired total energy, a desired power or a desired duration of the pulse.

13. The apparatus according to claim 7, wherein the bias circuitry comprises a transistor and/or switch, and the controller controls operation of the transistor and/or switch to provide the time-varying bias signal.

14. The apparatus according to claim 1, wherein the amplifier comprises negative and positive bias inputs, the bias circuitry is configured to provide a negative voltage to the negative input and a positive voltage to the positive input, and the bias circuitry and/or controller is configured to offset the application of the negative and positive voltages to the inputs, optionally so that the negative voltage is removed/reduced after and/or applied/increased before the positive voltage.

15. The apparatus according to claim 1, wherein the amplifier forms part of a gain stage and the apparatus comprises a further gain stage connected to the gain stage and comprising a further amplifier and further bias circuitry, and the controller is configured to control operation of the further bias circuitry to provide a time-varying bias signal thereby to provide a desired time variation of an output signal from the further amplifier.

16. The apparatus according to claim 1, wherein the controller is further configured to control power to at least one further component or device, and to switch off or reduce power to such at least one further component or device in a power saving mode, and to provide higher, operational power to the at least one further component or device in an operational mode.

17. The apparatus according to claim 16, wherein, in the operational mode, the controller is configured to reduce or switch off the bias signal during time periods when no output signal is desired thereby to reduce power consumption in the operational mode.

18. ( The apparatus according to claim 1, wherein the controller is configured to control the bias signal in dependence on input from a sensor and/or in dependence on input from a user.

19. The apparatus according to claim 18, wherein the controller is configured to reduce or switch off the bias signal to enter a low power mode in response to the sensor and/or user input.

20. The apparatus according to claim 1, wherein the controller controls the time-varying bias signal to reduce heat and/or thermal burden, by reducing or preventing overheating.

21. The apparatus according to claim 1, being a medical apparatus for providing treatment to a patient or other subject.

22. The apparatus according to claim 1, comprising or being for connection to a display device and or analogue-to-digital and/or digital-to-analogue converter.

23. The apparatus according to claim 1, wherein the output signal has power, or is such that the resulting electromagnetic radiation has power, in a range 1-50 W, optionally 8 W-10 W, 2 W-5 W or 3 W-8 W.

24. The apparatus according to claim 1, wherein the input signal has a frequency in a range of 0.1 GHz to 300 GHz.

25. The apparatus according to claim 1, wherein the controller is configured to control the bias signal to provide modulation of the output signal, with a modulation frequency in a range 1 Hz to 500 KHz, optionally wherein the modulation comprises amplitude modulation and/or pulse width modulation.

26. The apparatus according to claim 1, wherein the input signal is frequency modulated, optionally wherein the frequency modulation is in a range 1 to 500 KHz.

27. The apparatus according to claim 1, wherein the controller is configured to control the bias signal so that the output signal is modulated in accordance with a pulse width modulation (PWM) or an on/off keying (OOK) modulation scheme.

28. The apparatus according to claim 1, comprising an antenna or other radiating element that receives the output signal and outputs corresponding electromagnetic radiation.

29. An electromagnetic energy delivery system comprising:
a signal generator for generating r.f. or microwave or other signals having a desired frequency;
an apparatus according to any preceding claim configured so that the signals from the generator are provided to the amplifier input of the apparatus; and
a radiating element arranged to receive output signals from the amplifier output of the apparatus and to produce corresponding electromagnetic radiation.

30. A method of controlling operation of an electromagnetic energy delivery apparatus comprising controlling a bias signal applied to an amplifier of the apparatus thereby to provide a desired time variation of an output signal of the apparatus.
